(19) United States
(12) Patent Application Publication
(10) Pub. No.: US 2026/0183312 A1
(43) Pub. Date: Jul. 2, 2026

(54) A ROOM-TEMPERATURE STABLE, UNBUFFERED FORMULATION OF ROCURONIUM BROMIDE INJECTION FOR INTRAVENOUS ADMINISTRATION AND A METHOD THEREOF

(71) Applicant: MAIVA PHARMA PRIVATE LIMITED, Hosur (IN)

(72) Inventors: Alagumurugan Alagarswamy, Hosur (IN); Bhaskar Krishna Arumugam, Bangalore North (IN); Senthilkumar Natarajan, Hosur (IN); Swetha Ramasamy, Devakottai (IN)

(21) Appl. No.: 18/847,338

(22) PCT Filed: Nov. 3, 2023
(86) PCT No.: PCT/IB2023/061101

§ 371 Date: Sep. 16, 2024

(30) Foreign Application Priority Data
May 2, 2023 (IN) 202341031271
Nov. 3, 2023 (WO) PCT/IB2023/061101

(51) Int. Cl. A61K 31/58; A61K 9/00; A61K 9/08; A61K 47/02; A61K 47/26

(52) U.S. Cl. CPC A61K 31/58; A61K 9/0019; A61K 9/08; A61K 47/02; A61K 47/26

(57) ABSTRACT

According to an embodiment of the present disclosure, the room-temperature stable, unbuffered rocuronium injection formulation for intravenous administration comprises of 1% w/v of rocuronium bromide, a pH adjusting agent comprising hydrochloric acid at a concentration between 12.5 mMol and 20 mMol for the adjustment of pH between 3.5 to 4.5 and a quantity sufficient of water to make the formulation to 1 mL. The proposed formulation of the present disclosure is stabilized through optimization of pH of the formulation using only hydrochloric acid and/or sodium hydroxide as the pH adjusting agents without use of any buffering agents is suitable for moist heat sterilization. Further, the rocuronium injection formulation of the present disclosure contains lower than 3% of rocuronium related compound C, more preferably less than 2% over the stability period at room temperature conditions.

### EARLIEST PRIORITY DATE

This application claims priority from a patent application filed in India having patent application No. 202341031271, filed on May 2, 2023, and titled “A ROOM-TEMPERATURE STABLE, UNBUFFERED FORMULATION OF ROCURONIUM BROMIDE INJECTION FOR INTRAVENOUS ADMINISTRATION AND A METHOD THEREOF.” and a PCT Application no. PCT/IB2023/061101 filed on Nov. 3, 2023, and titled “A ROOM-TEMPERATURE STABLE, UNBUFFERED FORMULATION OF ROCURONIUM BROMIDE INJECTION FOR INTRAVENOUS ADMINISTRATION AND A METHOD THEREOF.”

### TECHNICAL FIELD

The present subject matter generally relates to pharmaceutical formulations of Rocuronium bromide injection. More specifically, the development of a stable and sterile rocuronium injection formulation having a preferred pH between 3.5 and 4.5 suitable for intravenous administration, without using any buffering agents, and capable of long-term storage at room temperature and amenable for moist heat sterilization.

### BACKGROUND OF THE INVENTION

Rocuronium bromide is a popular amino-steroidal non-depolarizing neuromuscular blocking agent with a quick onset of action and intermediate duration of blocking effects. The Reference products, Zemuron®/Esmeron® is a sterile, nonpyrogenic, clear, colorless to yellow/orange isotonic solution intended for intravenous injection.

The formulation of the Reference products (Zemuron®/Esmeron®) is buffered with an acetate buffer (approx. 140 mMol), wherein each mL contains 10 mg rocuronium bromide, 2 mg sodium acetate, and approximately 7 mg of Glacial acetic acid and the pH is adjusted to 4 with acetic acid and/or sodium hydroxide. Further, the aqueous solution is adjusted to isotonicity with sodium chloride.

Rocuronium is unstable in aqueous solutions and undergoes hydrolytic degradation to form primarily the deacetylated impurity called Rocuronium Related compound C. It may be further noted that the reference products Zemuron® or Esmeron® are recommended to be stored in a refrigerator at 2° C. to 8° C. (36° to 46° F.). It is further to be noted that upon the removal from the refrigeration condition, the Rocuronium bromide is to be used within 60 days to 12 weeks and the opened vials of rocuronium bromide within 30 days.

Various efforts have been made to improve the stability of rocuronium in an aqueous solution.

WO 2008/065142 discloses a technique to stabilize a rocuronium-containing aqueous solution by adding, to the solution, a sulfoalkyl ether-β-cyclodextrin derivative or a pharmaceutically acceptable salt thereof. However, this technique requires a sulfoalkyl ether-β-cyclodextrin derivative or pharmaceutically acceptable salt thereof, of which use has been reported to cause renal dysfunction, etc. The prior art document further describes that the pH should be in the range of 3.5 to 7.5, and preferably 5.5 to 7.5 for reducing injection pain. In other words, in the document, the occurrence of injection pain in the pH range of 3.5 to 7.5 is recognized and lowering the pH to 3.5 or less is not considered.

Rocuronium administration is associated with severe burning pain, which occurs in up to 80% of patients and lasts for approximately 10 to 20 seconds. Prior studies have found lidocaine, fentanyl, normal saline mixtures, dexmedetomidine, ketamine, and thiopental sodium to reduce rocuronium injection pain. However, studies indicate that a high concentration of acetate buffer used in the brand formulation, not the active ingredient, may be the cause of vascular pain.

U.S. Pat. No. 10,869,876 discloses a buffered rocuronium preparation with an adjusted pH of 3.5 or less (preferably between 2.8 to 3.2). The buffer solution may include citric acid-sodium hydroxide, tartaric acid-sodium hydroxide, potassium hydrogen phthalate-hydrochloric acid, or glycine-hydrochloric acid. Such a rocuronium preparation has a generation rate of Rocuronium-Related substance C of 5% or less after six months of storage at 40° C.

PCT patent WO2023275157 discloses an aqueous, room-temperature stable composition comprising rocuronium, wherein the composition has a pH of 2.5 to 3.5, preferably a pH of 2.8 to 3.2, most preferred a pH of 3.0, and wherein the composition has a titratable acidity of not more than 35 mEq. The rocuronium composition is stable upon thermal sterilization. Further, the rocuronium composition is stable upon storage at room-temperature.

However, such low pH of about 3 disclosed in the prior arts is not preferred generally due the acidic pH considering vascular irritation and injection pain. To keep the risk of local irritation low, pH values should nevertheless be inside the target pH range of 3.5≤pH≤9.0 unless for very compelling reason.

Other patent prior art disclosures, such as CN101653412B employ methods for stabilizing rocuronium using EDTA and buffering agent. The prior art EP2900216B1 discloses the composition of rocuronium salt with an excipient selected from D-gluconic acid with buffering agents.

There is still a need for a room-temperature stable rocuronium injection preparation suitable for intravenous administration having pH≥3.5 and closer to Reference products (i.e. pH about 4) but without use of acetate buffer or any buffering agents and still amenable for terminal sterilization by moist heat sterilization.

In order to address the above challenges, the inventors of the present disclosure found a solution by optimizing the composition of the injection, allowing for long-term storage at room temperature without the use of acetate or any other buffering agents and having pH closer to Reference products, Zemuron®/Esmeron®.

### SUMMARY OF THE INVENTION

In the light of the disadvantages mentioned in the previous section, the following summary is provided to facilitate an understanding of some of the innovative features unique to the present invention and is not intended to be a full description. A full appreciation of the various aspects of the invention can be gained by taking the entire specification and drawings as a whole.

In one embodiment, a room-temperature stable unbuffered rocuronium injection formulation for intravenous administration is disclosed. The formulation comprises of 1% w/v of rocuronium bromide, 0.7% to 0.9% w/v of tonicity adjusting agent, a pH adjusting agent, wherein the pH adjusting agent comprises hydrochloric acid at a concentration between 12.5 mMol and 20 mMol for the adjustment of pH between 3.5 to 4.5 and a quantity sufficient of water to make the formulation to 1 mL.

According to an embodiment of the present disclosure, the tonicity adjusting agent is selected from sodium chloride, mannitol, dextrose, and sucrose, preferably sodium chloride in the range of 0.7% to 0.9% w/v.

According to an embodiment of the present disclosure, the concentration of the hydrochloric acid is preferably in the range of 12.5 mMol to 20 mMol for pH adjustment between 3.5 to 4.5, preferably between pH 3.6 to 4.3 and most preferably between 3.6 to 3.8.

According to an embodiment of the present disclosure, the pH adjusting agent may also comprises sodium hydroxide, to be used if required to adjust the pH to a preferred range.

According to an embodiment of the present disclosure, the formulation comprises lower than 3% of rocuronium related compound C, more preferably less than 2% over the stability period at room temperature conditions.

In another embodiment, a method for preparing a room-temperature stable unbuffered rocuronium injection formulation for intravenous administration is disclosed. The method comprises of collecting water for injection and maintaining at a temperature range of 20-25° C., adding sodium chloride to the water and mixing for 10 minutes to dissolve, adding 0.5N hydrochloric acid solution, equivalent to 2.5% of batch volume, then adding rocuronium bromide and stirring for 20 minutes to dissolve, checking the pH and further adjusting the pH between 3.5 to 4.5, most preferably between 3.6 to 3.8 using 0.1N HCl solution and/or 0.1N NaOH solution, if required, making up the volume to 100% of batch size using water for injection and stirring for 20 minutes, aseptically filtering the solution through 0.2 μm sterilizing grade filters, filling approximately 5.3 mL of the filtered solution in 5 mL USP Type 1 flint tubular vials and crimping the vials with 13 mm aluminum flip-top seals. The vials may be additionally subjected to moist heat sterilization at 121.5° C. for 15 minutes, if desired.

The fill volume and vial size of the product may be varied according to the required dose, for example 10 mg/1 mL fill in a 2 mL vial, 20 mg/2 mL fill in a 2 mL vial, 50 mg/5 ml fill in 5 mL or 100 mg/10 mL fill in 10 mL vial. Similarly, the moist heat sterilization cycle may be changed appropriately to reduce overall thermal exposure of the vials, for example moist heat sterilization at 121° C. for 8 minutes exposure etc.

According to an embodiment of the present disclosure, the water for injection is sparged with nitrogen throughout the process except during the addition of the materials.

According to an embodiment of the present disclosure, the sterilizing grade filters used for aseptically filtering solution comprises of a pore size of 0.2 μm.

According to an embodiment of the present disclosure, the USP Type 1 flint tubular vials comprises a 13 mm neck and are stoppered with 13 mm West 4031/45 Bromobutyl stoppers or any alternate stoppers and crimped with 13 mm Aluminum flip-top seals.

According to an embodiment of the present disclosure, the pH of the solution is adjusted using 0.1N HCl solution and if required additionally using 0.1N NaOH solution to obtain a pH between 3.5 to 4.5, preferably between 3.6 and 4.3 and most preferably between 3.6 to 3.8.

### BRIEF DESCRIPTION OF THE ACCOMPANYING DRAWINGS

The detailed description is provided with reference to the accompanying figures. These and other features and advantages of the present invention will be readily appreciated, as the same becomes better understood by reference to the following detailed description when considered in connection with the accompanying drawings wherein:

FIG. 1 illustrates a method for preparing a room-temperature stable, unbuffered rocuronium injection formulation for intravenous administration in accordance with an embodiment of the present invention.

FIG. 2 is a graph illustrating the comparative levels of related compound C in the control formulation and the proposed formulation in accordance with an embodiment of the present invention at controlled room temperature conditions.

FIG. 3 is a graph illustrating the shift in product pH with moist heat sterilization at 121° C. for 15 min accordance with an embodiment of the present invention.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more devices or sub-systems or elements or structures or components preceded by “comprises” a″ does not, without more constraints, preclude the existence of other devices, sub-systems, elements, structures, components, additional devices, additional sub-systems, additional elements, additional structures, or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

As used herein, the term “Rocuronium bromide” refers to a medication used in anesthesia to help relax muscles during surgery or other medical procedures. It belongs to a class of drugs called neuromuscular blocking agents, which work by blocking the transmission of nerve impulses to the muscles, resulting in muscle relaxation and paralysis.

As used herein, the term “Room-temperature stable” refers to a substance or material that can be stored and maintained at normal room temperature (usually around 20-25° C. or 68-77° F.) without significant degradation or loss of quality over an extended period of time.

As used herein, the term “non-buffered” of “Unbuffered” refers to a substance or solution that does not contain a buffering agent. Buffers are commonly used in biological and chemical processes to prevent changes in pH that can affect the stability and activity of active substances, proteins, enzymes, and other molecules.

As used herein, the term “intravenous administration”, often abbreviated as IV administration, refers to the delivery of medication or fluids directly into a vein through a needle or catheter. This method of administration allows for the rapid and efficient delivery of medication or fluids into the bloodstream, bypassing the digestive system and allowing for more immediate effects.

Embodiments described herein disclose a room-temperature stable non-buffered rocuronium injection formulation for intravenous administration and a method thereof.

According to an embodiment of the present disclosure, the room-temperature stable non-buffered rocuronium injection formulation for intravenous administration comprises of 1% w/v of rocuronium bromide, a pH adjusting agent, wherein the pH adjusting agent comprises hydrochloric acid at a concentration between 12.5 mMol and 20 mMol for the adjustment of pH between 3.5 to 4.5, most preferably between 3.6 and 3.8 and a quantity sufficient of water to make the formulation to 1 mL.

According to an embodiment of the present disclosure, the tonicity adjusting agent is selected from sodium chloride, mannitol, dextrose, and sucrose, preferably sodium chloride in the range of 0.7% to 0.9% w/v.

According to an embodiment of the present disclosure, the concentration of the hydrochloric acid is preferably in the range of 12.5 mMol to 20 mMol for pH adjustment between 3.5 to 4.5, preferably between 3.6 to 4.3 and most preferably between 3.6 to 3.8.

According to an embodiment of the present disclosure, the pH adjusting agent may additionally comprises of sodium hydroxide, used when required for adjustment of pH.

According to an embodiment of the present disclosure, the formulation comprises lower than 3% of rocuronium related compound C, more preferably less than 2% over the stability period at room temperature conditions.

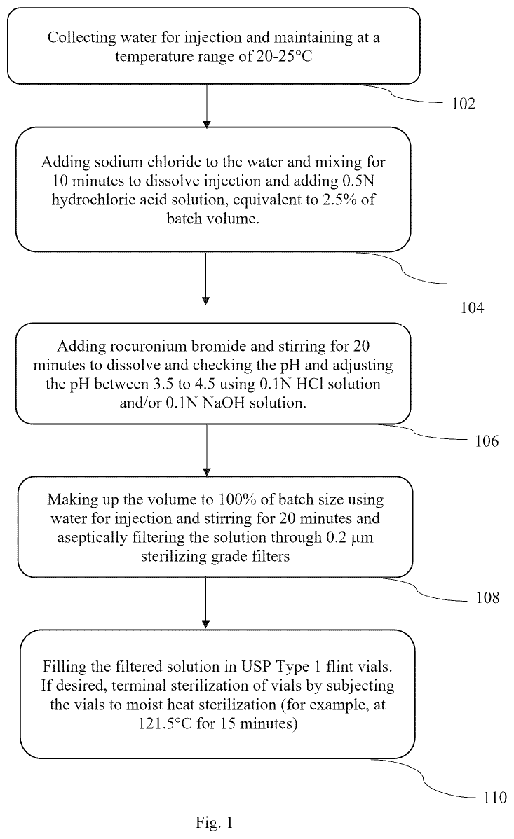

FIG. 1 illustrates a method for preparing a room-temperature stable unbuffered rocuronium injection formulation for intravenous administration in accordance with an embodiment of the present invention.

According to an embodiment of the present disclosure, the method for preparing a room-temperature stable unbuffered rocuronium injection formulation for intravenous administration comprises of collecting water for injection and maintaining at a temperature range of 20-25° C. at step 102. At step 104, adding sodium chloride to the water and mixing for 10 minutes to dissolve and adding 0.5N hydrochloric acid solution, equivalent to 2.5% of batch volume. At step 106, adding rocuronium bromide and stirring for 20 minutes to dissolve and checking the pH and further adjusting the pH between 3.5 to 4.5 using 0.1N HCl solution and/or 0.1N NaOH solution, if required. At step 108, making up the volume to 100% of batch size using water for injection and stirring for 20 minutes and aseptically filtering the solution through 0.2 μm sterilizing grade filters. Finally at step 110, filling approximately 5.3 mL of the filtered solution in 5 mL USP Type 1 flint tubular vials and crimping the vials with 13 mm aluminum flip-top seals. The filled vials may additionally be subjected to moist heat sterilization at 121.5° C. for 15 minutes, if desired.

In the above described process, the fill volume and vial size of the product may be varied according to the required dose, for example 10 mg/l mL fill in a 2 mL vial, 20 mg/2 ml fill in a 2 mL vial, 50 mg/5 mL fill in 5 mL or 100 mg/10 mL fill in 10 mL vial. Similarly, the moist heat sterilization cycle may be changed appropriately to reduce overall thermal exposure of the vials, for example moist heat sterilization at 121° C. for 8 minutes exposure etc.

According to an embodiment of the present disclosure, the water for injection is sparged with nitrogen throughout the process except during the addition of the materials.

According to an embodiment of the present disclosure, the sterilizing grade filters used for aseptically filtering solution comprises of a pore size of 0.2 μm.

According to an embodiment of the present disclosure, the USP Type 1 flint tubular vials comprises a 13 mm neck and are stoppered with 13 mm West 4031/45 Bromobutyl stoppers or any alternate stopper and crimped with 13 mm Aluminum flip-top seals. Depend on the desired dose, the fill volume and vial size of the product may be varied.

According to an embodiment of the present disclosure, the pH of the solution is adjusted using 0.1N HCl solution and/or 0.1N NaOH solution, if required to obtain a pH between 3.5 to 4.5, preferably between 3.6 to 4.3 and most preferably between 3.6 and 3.8.

Overall, the formulation of the present disclosure comprises of 0.7% to 0.9% w/w of sodium chloride as the tonicity agents to render the formulation isotonic and to obtain an osmolarity of approximately 290 mOsmol/L.

On the other hand, the marketed RLD product and Zemuron®, the tonicity is adjusted using approximately 3.3 mg/mL sodium chloride to obtain an isotonic solution. The formulation of the Reference products (Zemuron®/Esmeron®) is buffered with an acetate buffer (approx. 140 mMol), wherein each mL contains 10 mg rocuronium bromide, 2 mg sodium acetate, and approximately 7 mg of Glacial acetic acid and the pH is adjusted to 4 with acetic acid and/or sodium hydroxide.

The comparative composition of the reference products Zemuron® or Esmeron® and the formulation of the present disclosure is summarized in Table 1.

TABLE 1
Comparative composition of Reference products and Formulation of Present Invention

| | | | Control Formulation | Claimed Formulation | |
|---|---|---|---|---|---|
| | | | similar to Reference | without any | |
| | | | products | buffering agent | |
| | | | Each mL contains | Each mL | |
| Sr. No. | Ingredient | Function | (mg/mL) | contains | % w/v |
| 1 | Rocuronium | Active | 10.0 mg | 10.0 mg | 1.00 |
| | bromide USP | substance | | | |
| 2 | Sodium | Tonicity | Approx. 3.3 mg | 8.0 mg | 0.80 |
| | Chloride USP | adjusting agent | | | |
| 3 | Sodium acetate | Buffering agent | 2.0 mg | Acetate buffer or any | |
| | anhydrous USP | | | other buffer is not | |
| 4 | Glacial Acetic | pH Adjusting | Approx. 7.0 mg | used in the | |
| | acid, USP | agents | (Note 1) | formulation | |
| 5 | Sodium hydroxide NF | | q.s. to adjust pH | q.s. to adjust pH | |
| | | | | between 3.5 to 4.5 | |
| 6 | Hydrochloric | pH Adjusting | Not used | (Note 2) | |
| | acid NF | agent | | | |

| | | | | |
|---|---|---|---|---|
| 7 | Water for | Vehicle | q.s. to 1 mL | q.s. to 1 mL |
| | Injection USP | | | |

From the above, it can be noted (Note 1) that the RLD, ZEMURON® formulation contains an acetate buffer comprising about 2.0 mg of sodium acetate and approx. 7 mg of Glacial acetic acid. Further, Glacial acetic acid and/or sodium hydroxide is used for pH adjustment if required to adjust the pH to about 4.

On the other hand, it can also be noted (Note 2) from the above that the proposed formulation of the present disclosure is unbuffered and does not contain acetate or other buffering agents. The pH of the formulation is adjusted using Hydrochloric acid and/or sodium hydroxide to about 3.5 to 4.5, preferably between 3.6 to 4.3 and most preferably between 3.6 to 3.8.

### Working Examples

### Method Steps for Preparing Control Formulation, Similar to Reference Products

Step 1. Collect Water for injection (about 90.0%) of batch size in the manufacturing vessel and maintain at a temperature of 20-25° C. Sparge the nitrogen throughout the process except during the material addition.
Step 2. Add batch quantity of sodium chloride and mix for 10 minutes to dissolve and obtain a clear solution.
Step 3. Add batch quantity of Sodium acetate anhydrous and stir for 10 minutes to dissolve and obtain a clear solution.
Step 4. Add batch quantity of Glacial acetic acid (equivalent to 7 mg/mL) and stir for 10 minutes to obtain a clear solution.
Step 5. Add batch quantity of Rocuronium Bromide and stir for 20 minutes to dissolve and obtain a clear solution.
Step 6. Check the pH and if required adjust the pH to 4.0±0.1 using 10% acetic acid and/or 0.1N NaOH solution.
Step 7. Make up the volume to 100% of batch size using the Water for injection and stir for 20 minutes.
Step 8. Aseptically filter the solution through 0.2 μm sterilizing grade filters and fill Approx. 5.3 mL filtered solution in 5 mL USP Type 1 flint tubular vials with 13 mm neck. Stopper the vials with 13 mm West 4031/45 Bromobutyl stoppers and crimp the vials with 13 mm Aluminum flip-top seals.

### Method Steps for Claimed Formulation of the Present Disclosure

Step 1: Collect Water for injection (about 90.0%) of batch size in the manufacturing vessel and maintain at a temperature of 20-25° C. Sparge the nitrogen throughout the process except during the material addition.
Step 2: Add batch quantity of sodium chloride and mix for 10 minutes to dissolve and obtain a clear solution.
Step 3: Add 0.5N hydrochloric acid solution, a quantity equivalent to 2.5% of batch volume. This hydrochloric acid quantity is equivalent to approx. 12.5 mMol.
Step 4: Add batch quantity of Rocuronium bromide slowly and stir for 20 minutes to dissolve and obtain a clear solution.
Step 5: Check the pH and if required adjust the pH between 3.5 to 4.5, preferably between 3.6 to 4.3 and most preferably between 3.6 to 3.8 using 0.1N HCl solution and/or 0.1N NaOH solution, if required.
Step 6: Make up the volume to 100% of batch size using the Water for injection and stir for 20 minutes,
Step 7: Aseptically filter the solution through 0.2 μm sterilizing grade filters and fill Approx. 5.3 mL filtered solution in 5 mL USP Type 1 flint tubular vials with 13 mm neck. Stopper the vials with 13 mm West 4031/45 Bromobutyl stoppers and crimp the vials with 13 mm Aluminum flip-top seals.
Step 8: If desired, terminally sterilize the product by subjecting the vials to moist heat sterilization, for example at 121.5° C. for 15 minutes.

### Results

The room temperature (25° C./60% RH) stability of the control formulation (formula similar to marketed Reference products) is provided in Table 2.

TABLE 2
Stability profile of the Aseptically filtered, Control Formulation
in room temperature (25° C./60% RH) conditions.

| | | | Aseptically | Controlled Room | |
|---|---|---|---|---|---|
| | | | filtered | temperature condition | |
| | | Acceptance | solution | (25° C./60% RH) | |
| S. No. | Test Parameters | Criteria | (Initial) | 3M | 6M |
| 1 | Description | A Clear colorless to | Complies | Complies | Complies |
| | | pale yellow solution | | | |
| | | free from visible | | | |
| | | particles. | | | |
| 2 | pH | Between 3.5 to 4.5 | 4.08 | 4.09 | 4.11 |
| 3 | Osmolality | For information | 325 | 325 | 331 |
| | (mOsm/kg) | | | | |
| 4 | Assay of | NLT 90.0% and | 101.4 | 99.8 | 100.1 |
| | Rocuronium | NMT 110.0% | | | |
| | Bromide | | | | |
| 5 | Related substances by HPLC | | | | |
| | Rocuronium related | NMT 1.5% | 0.12 | 0.68 | 1.39 |
| | compound C | | | | |
| | Any Individual Impurity | NMT 0.2% | BDL | 0.14 | 0.13 |
| | Total Impurities | NMT 3.0% | 0.12 | 0.82 | 1.57 |

The stability of the claimed formulation of the present disclosure in controlled room temperature (25° C./60% RH) is provided in Table 3.

TABLE 3
Stability profile of the proposed formulation in controlled
room temperature (25° C./60% RH) conditions

| | | | | Controlled Room temperature | | | |
|---|---|---|---|---|---|---|---|
| | | | | conditions (25° C./60% RH) | | | |
| | | | | Terminally | | | |
| | | | Aseptically | sterilized | | | |
| | Test | Acceptance | filled vials | product- | | | |
| S. No. | Parameters | Criteria | (Before TS) | Initial (T0) | 3 Month | 6 Month | 12 Month |
| 1 | Appearance | A clear | Complies | Complies | Complies | Complies | Complies |
| | | colorless to | | | | | |
| | | pale yellow | | | | | |
| | | solution free | | | | | |
| | | from visible | | | | | |
| | | particles. | | | | | |
| 2 | pH | Between | 3.66 | 3.57 | 3.67 | 3.65 | 3.68 |
| | | 3.5 and 4.3 | | | | | |
| 3 | Osmolality | Between | 269 | 271 | 267 | 260 | 270 |
| | (mOsm/kg) | 250 to 340 | | | | | |
| 4 | Assay | NLT 90.0% | 100.8 | 99.5 | 102.9 | 101.3 | 99.6 |
| | | and NMT | | | | | |
| | | 110.0% | | | | | |
| 5 | Related Substances test | | | | | | |
| | Rocuronium | NMT 3.0% | 0.36 | 0.41 | 0.66 | 0.76 | 1.02 |
| | related | | | | | | |
| | compound C | | | | | | |
| | Any Individual | NMT 0.2% | 0.08 | 0.11 | 0.18 | BDL | 0.05 |
| | Impurity | | | | | | |
| | Total | NMT 3.0% | 0.59 | 0.78 | 1.13 | 0.85 | 1.18 |
| | Impurities | | | | | | |

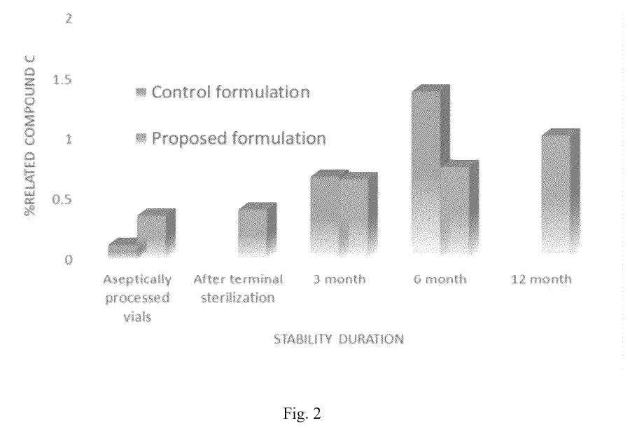

FIG. 2 is a graph illustrating the levels of Related compound C in the control formulation and the proposed formulation in accordance with an embodiment of the present invention. The data demonstrate that the proposed formulation of the present disclosure is found be significantly stable and is suitable for long term storage in room temperature conditions in comparison to the control formulation.

Further, the stability profile of the formulations with the proposed composition as per the proposed formulation at different pH across the desired pH range was assessed and is provided in Table 4.

### Table 4. Comparative Stability Profile of the Claimed Formulation in Controlled Room Temperature (25° C./60% RH) Conditions

TABLE 4a
Formulation with pH about 3.7

| | | Formulation with pH about 3.7 | | | |
|---|---|---|---|---|---|
| | | | Vials after | 1 Month | 1 Month |
| | | Aseptically | Terminal | (40° C./75% | (25° C./60% |
| Test Parameters | Limited | filled vials | sterilization | RH) | RH) |
| Description | A clear colorless to pale | Complies | Complies | Complies | Complies |
| | yellow solution free | | | | |
| | from visible particles | | | | |
| pH | 3.5-4.5 | 3.72 | 3.68 | 3.7 | 3.73 |
| Assay of Rocuronium | NLT 90.0%- | 106 | 102.9 | 100.3 | 100 |
| Bromide | NMT 110.0% | | | | |

Other Impurities

| | | | | | |
|---|---|---|---|---|---|
| Rocuronium related | NMT 3.5% | 0.11 | 0.22 | 0.64 | 0.5 |
| compound C | | | | | |
| Any individual impurity | NMT 0.2% | BDL | BDL | BDL | BDL |
| Total Impurities | NMT 4.5% | 0.11 | 0.22 | 0.64 | 0.5 |

TABLE 4b
Formulation with pH about 4.0

| | | Formulation with pH about 4.0 | | | |
|---|---|---|---|---|---|
| | | | Vials after | 1 Month | 1 Month |
| | | Aseptically | Terminal | (40° C./75% | (25° C./60% |
| Test Parameters | Limited | filled vials | sterilization | RH) | RH) |
| Description | A clear colorless to pale | Complies | Complies | Complies | Complies |
| | yellow solution free | | | | |
| | from visible particles | | | | |
| pH | 3.5-4.5 | 3.88 | 3.94 | 3.98 | 4.02 |
| Assay of Rocuronium | NLT 90.0%- | 104.9 | 102.9 | 99.7 | 100.6 |
| Bromide | NMT 110.0% | | | | |

Other Impurities

| | | | | | |
|---|---|---|---|---|---|
| Rocuronium related | NMT 3.5% | 0.11 | 0.23 | 0.78 | 0.4 |
| compound C | | | | | |
| Any individual impurity | NMT 0.2% | BDL | BDL | BDL | BDL |
| Total Impurities | NMT 4.5% | 0.11 | 0.23 | 0.78 | 0.4 |

TABLE 4c
Formulation with pH about 4.2

| | | Formulation with pH about 4.2 | | | |
|---|---|---|---|---|---|
| | | | Vials after | 1 Month | 1 Month |
| | | Aseptically | Terminal | (40° C./75% | (25° C./60% |
| Test Parameters | Limited | filled vials | sterilization | RH) | RH) |
| Description | A clear colorless to pale | Complies | Complies | Complies | Complies |
| | yellow solution free | | | | |
| | from visible particles | | | | |
| pH | 3.5-4.5 | 4.17 | 4.22 | 4.16 | 4.2 |
| Assay of Rocuronium | NLT 90.0%- | 105.1 | 103.1 | 100.2 | 100.9 |
| Bromide | NMT 110.0% | | | | |

Other Impurities

| | | | | | |
|---|---|---|---|---|---|
| Rocuronium related | NMT 3.5% | 0.11 | 0.23 | 0.7 | 0.49 |
| compound C | | | | | |
| Any individual impurity | NMT 0.2% | BDL | BDL | BDL | BDL |
| Total Impurities | NMT 4.5% | 0.11 | 0.23 | 0.7 | 0.49 |

The stability trend data demonstrates that the product is stable at the desired pH range. There was no significant change in parameters such as pH, osmolarity, potency observed over the stability evaluation. The stability trend of Related compound C was found comparable across the desired pH range.

Further, the trend data demonstrates that the formulation of the present disclosure is suitable for terminal sterilization as there is no significant change observed when the product is sterilized by moist heat sterilization at 121° C. for 15 minutes.

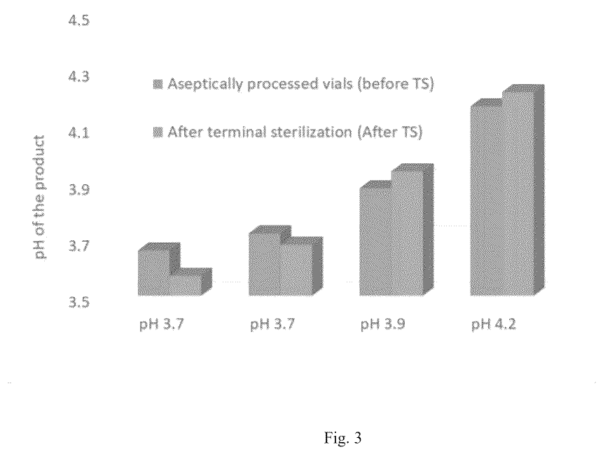

FIG. 3 is a graph illustrating the effect of moist heat sterilization at 121° C. for 15 minutes on the pH of the formulation having different pH across the desired pH range in accordance with an embodiment of the present invention. The data demonstrate that there is no significant shift in the pH with the moist heat terminal sterilization. Various embodiments of the room-temperature stable, unbuffered rocuronium injection formulation for intravenous administration and the method thereof described above enable various advantages. The proposed formulation of the present disclosure is stabilized through optimization of pH of the formulation using only hydrochloric acid and/or sodium hydroxide as the pH adjusting agents without use of any buffering agents. Through this optimization, the inventors of the present disclosure found a surprising technical advancement in improved stability suitable for moist heat sterilization and long-term storage at room temperature without use of any acetate or any other buffering agents.

Further, the rocuronium injection formulation of the present disclosure contains lower than 3% of rocuronium related compound C, more preferably less than 2% over the stability period at room temperature conditions.

It will be understood by those skilled in the art that the foregoing general description and the following detailed description are exemplary and explanatory of the disclosure and are not intended to be restrictive thereof. While specific language has been used to describe the disclosure, any limitations arising on account of the same are not intended.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, the order of processes described herein may be changed and are not limited to the manner described herein. Moreover, the actions of any flow diagram need not be implemented in the order shown; nor do all the acts need to be necessarily performed. Also, those acts that are not dependent on other acts may be performed in parallel with the other acts. The scope of embodiments is by no means limited by these specific examples.

We claim:

1. A room-temperature stable, unbuffered rocuronium bromide injection formulation for intravenous administration having a pH between 3.5 and 4.5, wherein the formulation comprising:
a) 1% w/v of rocuronium bromide;
b) 0.7% to 0.9% w/v of tonicity adjusting agent;
c) concentration of pH adjusting agent, hydrochloric acid maintained between 12.5 mMol and 20 mMol;
d) a quantity sufficient of water to make the formulation to 1 mL.

2. The formulation as claimed in claim 1, wherein the tonicity adjusting agent is selected from sodium chloride, mannitol, dextrose, and sucrose, preferably sodium chloride in the range of 0.7% to 0.9% w/v.

3. The formulation as claimed in claim 1, wherein the concentration of the hydrochloric acid is preferably in the range of 12.5 mMol to 20 mMol for pH adjustment between 3.5 to 4.5.

4. The formulation as claimed in claim 1, wherein the pH adjusting agent may additionally comprises sodium hydroxide, to be used if required for adjustment of pH.

5. The formulation as claimed in claim 1, wherein the formulation comprises lower than 3% of rocuronium related compound C, more preferably less than 2% over the stability period at room temperature conditions.

6. A method for preparing a room-temperature stable unbuffered rocuronium bromide injection formulation for intravenous administration, the method comprises:
a) collecting water for injection and maintaining at a temperature range of 20-25° C.;
b) adding sodium chloride to the water and mixing for 10 minutes to dissolve;
c) adding 0.5N hydrochloric acid solution, equivalent to 2.5% of batch volume;
d) adding rocuronium bromide and stirring for 20 minutes to dissolve;
e) checking the pH and adjusting the pH between 3.5 to 4.5 using 0.1N HCl solution and/or 0.1N NaOH solution;
f) making up the volume to 100% of batch size using water for injection and stirring for 20 minutes;
g) aseptically filtering the solution through 0.2 μm sterilizing grade filters;
h) filling the filtered solution in vials and crimping the vials with aluminum flip-top seals; and
i) if desired, subjecting the vials additionally to moist heat sterilization, for example at 121.5° C. for 15 minutes.

7. The method as claimed in claim 6, wherein the water for injection is sparged with nitrogen throughout the process except during the addition of the materials.

8. The method as claimed in claim 6, wherein the sterilizing grade filters used for aseptically filtering solution comprises of a pore size of 0.2 μm.

9. The method as claimed in claim 6, wherein the USP Type 1 flint tubular vials comprises a 13 mm neck and are stoppered with 13 mm West 4031/45 Bromobutyl stoppers or any alternate stoppers and crimped with 13 mm Aluminum flip-top seals.

10. The method as claimed in claim 6, wherein the pH of the solution is adjusted using 0.1N HCl solution and/or 0.1N NaOH solution to obtain a pH between 3.5 to 4.5, preferably between 3.6 and 4.3 and most preferably between 3.6 and 3.8.
